(19) United States
(12) Patent Application Publication
(10) Pub. No.: US 2024/0175873 A1
(43) Pub. Date: May 30, 2024

(54) METHODS FOR THE DIAGNOSIS AND TREATMENT OF T CELL-LYMPHOMAS

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS, Paris (FR); UNIVERSITÉ PARIS CITÉ, Paris (FR); UNIVERSITÉ PARIS-EST CRÉTEIL VAL DE MARNE, Creteil (FR)

(72) Inventors: Armand BENSUSSAN, Paris (FR); Jérôme GIUSTINIANI, Creteil (FR); Martine BAGOT, Paris (FR); Adèle DE MASSON D'AUTUME, Creteil (FR); Nicolas ORTONNE, Creteil (FR); Maxime BATTISTELLA, Paris Cedex 10 (FR); Anne MARIE-CARDINE, Paris Cedex 10 (FR)

(21) Appl. No.: 18/282,667

(22) PCT Filed: Mar. 22, 2022
(86) PCT No.: PCT/EP2022/057408

§ 371 Date: Sep. 18, 2023

(30) Foreign Application Priority Data
Mar. 23, 2021 (EP) 21305356.4
Dec. 10, 2021 (EP) 21306740.8

(51) Int. Cl. G01N 33/574; A61K 35/17; A61K 39/00; A61K 47/68; C07K 16/28

(52) U.S. Cl. CPC G01N 33/57426; A61K 35/17; A61K 39/4611; A61K 39/4631; A61K 39/464421; A61K 47/6849; C07K 16/2866; A61K 2039/505; C07K 2317/31; C07K 2317/732

(57) ABSTRACT

T-cell lymphomas are a heterogeneous group of malignancies involving T lymphocytes and generally characterized by a poor prognosis. Among them, cutaneous T-cell lymphomas involve primarily the skin. Mycosis fungoides and Sezary syndrome are the most frequent cutaneous T-cell lymphomas. The inventors studied the regulatory T phenotype of Sezary cells and showed the expression of CCR8 (CD198) by Sezary cells and other T-cell lymphoma cell lines. CCR8 therefore appears as a useful diagnostic, prognostic and follow-up marker, and as a potential therapeutic target in T-cell lymphomas. Therapeutic depletion of CCR8-expressing cancer cells would eliminate tumor cells and also activate the anti-tumor immunity in T-cell lymphomas.

### FIELD OF THE INVENTION

The present invention is in the field of medicine, in particular oncology.

### BACKGROUND OF THE INVENTION

T-cell lymphomas are a heterogeneous group of malignancies involving T lymphocytes, and generally characterized by a poor prognosis. Among them, cutaneous T-cell lymphomas (CTCL) involve primarily the skin. Mycosis fungoides and Sézary syndrome (SS) are the most frequent CTCL. The circulating clonal tumor T cells (Sézary cells) express CD4 and may lose expression of CD7 and CD26, while exhibiting in most cases aberrant expression of CD158k (KIR3DL2) (1,2). Long-term responses are rare in advanced-stage CTCL and new treatments are needed. Recently, treatment with anti-CCR4 monoclonal antibody (mogamulizumab) has improved progression-free survival in CTCL (3). CCR4 is expressed not only by Sézary cells but also by peripheral blood activated regulatory T cells (Treg) (4), and depletion of CCR4+ circulating Treg by mogamulizumab treatment is associated with the occurrence of autoimmune adverse events (5,6). Besides CCR4, Sézary cells express several Treg markers and immune checkpoint inhibitors, such as PD1 (7), CD39 (8) and TIGIT (9). The expression of these markers by Sézary cells led us to investigate the expression of CCR8 (CD198), a chemokine receptor involved in the homing of lymphocytes to the skin (10).

### SUMMARY OF THE INVENTION

The present invention is defined by the claims. In particular, the present invention relates to methods for the diagnosis and treatment of T cell-lymphomas.

### DETAILED DESCRIPTION OF THE INVENTION

The inventors studied the regulatory T phenotype of Sézary cells and showed the expression of CCR8 (CD198) by Sézary cells and other T-cell lymphoma cell lines. CCR8 is a chemokine receptor involved in the homing of lymphocytes to the skin (10). CCR8 is expressed by skin resident memory T cells (T_{RM}) (11) which are suspected to be the tumor cells of origin in mycosis fungoides (12). CCR8 is also strongly expressed by tumor-infiltrating regulatory T cells involved in immune escape while expression on peripheral blood Treg was lower (13). Depletion of CCR8+ Treg exerted a strong antitumor effect independently or in combination with PD-1 inhibitor in LLC-OVA and MC38 tumor mouse model (13). CCR8 therefore appears as a useful diagnostic, prognostic and follow-up marker, and as a potential therapeutic target in T-cell lymphomas. Therapeutic depletion of CCR8-expressing cancer cells would eliminate tumor cells and also activate the anti-tumor immunity in T-cell lymphomas.

### Main Definitions

As used herein, the term “T cell” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8+ T cells, CD4+ T cells, and gamma delta T cells. As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor infiltrating CD8+ T cell” refers to the pool of CD8+ T cells of the patient that have left the blood stream and have migrated into a tumor. As used herein, the term “CD4+ T cells” (also called T helper cells or TH cells) refers to T cells which express the CD4 glycoprotein on their surfaces and which assist other white blood cells in immunologic processes, including maturation of B cells into plasma cells and memory B cells, and activation of cytotoxic T cells and macrophages. CD4+ T cells become activated when they are presented with peptide antigens by MHC class II molecules, which are expressed on the surface of antigen-presenting cells (APCs). Once activated, they divide rapidly and secrete cytokines that regulate or assist in the active immune response. These cells can differentiate into one of several subtypes, including TH1, TH2, TH3, TH17, TH9, TFH or Treg, which secrete different cytokines to facilitate different types of immune responses. Signaling from the APC directs T cells into particular subtypes. In addition to CD4, the TH cell surface biomarkers known in the art include CXCR3 (Th1), CCR4, Crth2 (Th2), CCR6 (Th17), CXCR5 (Tfh) and as well as subtype-specific expression of cytokines and transcription factors including T-bet, GATA3, EOMES, RORγT, BCL6 and FoxP3. As used herein, the term “gamma delta T cell” has its general meaning in the art. Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MHC molecules of antigen-presenting cells. Gamma 9 delta 2 T cells (sometimes also called gamma 2 delta 2 T cells) are gamma delta T cells bearing TCR receptors with the variable domains Vγ9 and Vδ2. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62: 1-5) or by other intracellular parasites, such as mycobacteria (Constant et al., Infection and Immunity, December 1995, vol. 63, no. 12: 4628-4633) or protozoa (Behr et al., Infection and Immunity, 1996, vol. 64, no. 8: 2892-2896). They may also be cancer cells (Poccia et al., J. Immunol., 159: 6009-6015; Fournie and Bonneville, Res. Immunol., 66th Forum in Immunology, 147: 338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

As used herein, the term “T-cell lymphoma” has its general meaning in the art and refers to a rare form of cancerous lymphoma affecting T-cells. Lymphoma arises mainly from the uncontrolled proliferation of T-cells and can become cancerous. T-cell lymphoma is categorized under Non-Hodgkin Lymphoma (NHL) and represents less than 15% of all Non-Hodgkin's diseases in the category. T-cell lymphomas are often categorised based on their growth patterns as either; aggressive (fast-growing) or indolent (slow-growing). In particular, T-cell lymphomas include peripheral T-cell lymphomas, Angioimmunoblastic T-cell lymphoma (AITL), Hepatosplenic T-cell lymphoma (HSTL), Natural Killer T-cell lymphoma (NKTL), and Cutaneous T-cell lymphoma (CTCL).

As used herein, the term “cutaneous T-cell lymphoma” or “CTCL” has its general meaning in the art and refers to a rare heterogeneous group of non-Hodgkin lymphomas derived from skin-homing mature T-cells. Mycosis fungoides (MF) and Sezary Syndrome (SS) represent the most common subtypes of primary CTCL, with an incidence rate of 4.1/1,000,000 person-years and male predominance.

As used herein the term “Sézary syndrome” or “SS” has its general meaning in the art and refers to an aggressive form of cutaneous T-cell lymphoma characterized by a triad of erythroderma, lymphadenopathy and circulating atypical lymphocytes (Sézary cells). SS develops most frequently in men, is more frequent in the elderly, and progresses rapidly. SS correspond to stages IVA2 and IVB of T-cell cutaneous lymphoma (see this term). Patients present with a scaling erythroderma and infiltration often manifesting with leonine facies and severe pruritus. Alopecia, ectropium, mild palmoplantar keratoderma and nail onychodystrophy may be present. Lymphadenopathy and hepatosplenomegaly are observed. Patients often shiver and complain of chills and general fatigue.

As used herein, the term “CCR8” has its general meaning in the art and refers to the C-C chemokine receptor type 8. The term is also named as CD198, CKRL1, CMKBR8, or CMKBRL2. An exemplary amino acid sequence for CCR8 is shown as SEQ ID NO:1. The receptor is characterized by several extracellular domains that are defined by the following positions 1-35, 94-107, 172-202, and 264-280 in SEQ ID NO:1.

>sp | P51685 | CCR8_HUMAN C-C chemokine
receptor type 8
OS = Homo sapiens OX = 9606 GN = CCR8
PE = 1 SV = 1
SEQ ID NO: 1
MDYTLDLSVTTVTDYYYPDIFSSPCDAELI
QTNGKLLLAVFYCLLFVFSLLGNSLVILVL
VVCKKLRSITDVYLLNLALSDLLFVFSFPF
QTYYLLDQWVFGTVMCKVVSGFYYIGFYSS
MFFITLMSVDRYLAVVHAVYALKVRTIRMG
TTLCLAVWLTAIMATIPLLVFYQVASEDGV
LQCYSFYNQQTLKWKIFTNFKMNILGLLIP
FTIFMFCYIKILHQLKRCQNHNKTKAIRLV
LIVVIASLLFWVPFNVVLFLTSLHSMHILD
GCSISQQLTYATHVTEIISFTHCCVNPVIY
AFVGEKFKKHLSEIFQKSCSQIFNYLGRQM
PRESCEKSSSCQQHSSRSSSVDYIL

As used herein, the term “agent capable of inducing cell death of CCR8 expressing cancer cells” refers to any molecule that under cellular and/or physiological conditions is capable of inducing cell death of CCR8 expressing cancer cells. In particular, the agent is capable of inducing apoptosis of CCR8 expressing cancer cells. In some embodiments, the agent is capable of depleting CCR8 cancer cells. As used herein, the term “depletion” with respect to cancer cells, refers to a measurable decrease in the number of CCR8 expressing cancer cells in the patient. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of CCR8 cancer cells in the patient below detectable limits.

As used herein, the term “CCR8 inhibitor” refers to a molecule that partially or fully blocks, inhibits, or neutralizes a biological activity or expression of CCR8. A CCR8 inhibitor can be a molecule of any type that interferes with the signalling associated with CCR8 in a cell, for example, either by decreasing transcription or translation of CCR8-encoding nucleic acid, or by inhibiting or blocking CCR8 polypeptide activity, or both. Examples of CCR8 inhibitors include, but are not limited to, antisense polynucleotides, interfering RNAs, catalytic RNAs, RNA-DNA chimeras, CCR8-specific aptamers, anti-CCR8 antibodies, CCR8-binding fragments of anti-CCR8 antibodies, CCR8-binding small molecules, CCR8-binding peptides, and other polypeptides that specifically bind CCR8 (including, but not limited to, CCR8-binding fragments of one or more CCR8 ligands, optionally fused to one or more additional domains), such that the interaction between the CCR8 inhibitor and CCR8 results in a reduction or cessation of CCR8 activity or expression.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404,097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

As used herein, the term “fully human” refers to an immunoglobulin, such as an antibody or antibody fragment, where the whole molecule is of human origin or consists of an amino acid sequence identical to a human form of the antibody or immunoglobulin.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

As used herein, the term “bispecific antibody” has its general meaning in the art and refers to an artificial, hybrid antibody having two different pairs of heavy and light chain and also two different antigen-binding sites.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. Moreover, when expressed in T-cells, CARs advantageously do not dimerize with endogenous T cell receptor (TCR) alpha and beta chains. The chimeric antigen receptor the present invention typically comprises an extracellular hinge domain, a transmembrane domain, and an intracellular T cell signaling domain.

As used herein the term “CAR-T cell” refers to a T lymphocyte that has been genetically engineered to express a CAR. The definition of CAR T-cells encompasses all classes and subclasses of T-lymphocytes including CD4+, CD8+ T cells, gamma delta T cells as well as effector T cells, memory T cells, regulatory T cells, and the like. The T lymphocytes that are genetically modified may be “derived” or “obtained” from the patient who will receive the treatment using the genetically modified T cells or they may “derived” or “obtained” from a different patient.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of drug are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of a inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled inhibitor of the present invention, fragment or mini-antibody derived from the inhibitor of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

### Methods of Treating

Accordingly, the first object of the present invention relates to a method of treating a T-cell lymphoma in a patient in need thereof comprising administering to the patient a therapeutically effective amount of an agent capable of inducing cell death of CCR8 expressing cancer cells.

In some embodiments, the T-cell lymphoma is angioimmunoblastic T-cell lymphoma, hepatosplenic T-cell lymphoma, natural killer T-cell lymphoma or cutaneous T-cell lymphoma. In some embodiments, the T-cell lymphoma is cutaneous T-cell lymphoma. More particularly, the T-cell lymphoma is Sézary syndrome.

In some embodiments, the patient is a human infant. In some embodiments, the patient is a human child. In some embodiments, the patient is a human adult. In some embodiments, the patient is an elderly human. In some embodiments, the patient is a premature human infant.

In some embodiments, the agent capable of inducing cell death of CCR8 expressing cancer cells is a CCR8 inhibitor.

CCR8 inhibitors are well-known in the art. As example, the CCR8 inhibitor may be AZ084 (Cas No. 929300-19-6), ML604086 (Cas No. 850330-18-6), R243 (Cas No. 688352-84-3), LMD-A (Cas No. 850330-77-7), MC148, CDBP0728 or CDBP5280. CCR8 inhibitor are also described in patent literature, e.g. WO/2004/058736.

### CCR8 Antibodies:

In some embodiments, the agent is an antibody having binding affinity for CCR8. In some embodiments, the agent is an antibody directed against at least one extracellular domain of CCR8. In some embodiments, the antibody is an anti-CCR8 neutralizing antibody. In some embodiments, the antibody inhibits CCL1, CCL8 or CCL18-mediated activation of CCR8. In some embodiments, the antibody leads to the depletion of CCR8 expression cancer cells.

In some embodiments, the antibody is a humanized antibody or a chimeric antibody.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody is obtainable from the hybridoma having ATCC Accession No. PTA-6940.

In some embodiments, the antibody is obtainable from the hybridoma having ATCC Accession No. PTA-6938.

In some embodiments, the antibody is obtainable from the hybridoma having ATCC Accession No. PTA-6939.

In some embodiments, the antibody is the HBM1022 antibody as disclosed in Lu S, Hu S, Gan X, et al711 HBM1022, a novel anti-CCR8 antibody depletes tumor-infiltrating regulatory T cells via enhanced ADCC activity, mediates potent anti-tumor activity with Keytruda. Journal for ImmunoTherapy of Cancer 2020; 8:doi: 10.1136/jitc-2020-SITC2020.0711

In some embodiments, the antibody is the FPA157 antibody as disclosed in Rankin A, Naik E861 Development of FPA157, an anti-CCR8 depleting antibody engineered to preferentially eliminate tumor-infiltrating T regulatory cells. Journal for ImmunoTherapy of Cancer 2020; 8:doi: 10.1136/jitc-2020-SITC2020.0861

In some embodiments, the antibody is the SRF114 antibody as disclosed in Lake A, Warren M, Das S, et al726 SRF114 is a fully human, CCR8 selective IgG1 antibody that induces destruction of tumor Tregs through ADCC. Journal for ImmunoTherapy of Cancer 2020; 8:doi: 10.1136/jitc-2020-SITC2020.0726

In some embodiments, the antibody is the anti-CCR8 hIgG1-nonfucosylated BMS-986340 antibody as disclosed in Lan, Ruth, et al. “Highly selective anti-CCR8 antibody-mediated depletion of regulatory T cells leads to potent antitumor activity alone and in combination with anti-PD-1 in preclinical models.” (2020): 6694-6694 and in Bayati F, Mohammadi M, Valadi M, Jamshidi S, Foma A M, Sharif-Paghaleh E. The Therapeutic Potential of Regulatory T Cells: Challenges and Opportunities. Front Immunol. 2021; 11:585819. Published 2021 Jan. 15. doi:10.3389/fimmu.2020.585819

In some embodiments, the antibody is the nanobody as disclosed in Van Damme H, Dombrecht B, Kiss M, Roose H, Allen E, Van Overmeire E, Kancheva D, Martens L, Murgaski A, Bardet P M R, Blancke G, Jans M, Bolli E, Martins M S, Elkrim Y, Dooley J, Boon L, Schwarze J K, Tacke F, Movahedi K, Vandamme N, Neyns B, Ocak S, Scheyltjens I, Vereecke L, Nana F A, Merchiers P, Laoui D, Van Ginderachter J A. Therapeutic depletion of CCR8+ tumor-infiltrating regulatory T cells elicits antitumor immunity and synergizes with anti-PD-1 therapy. J Immunother Cancer. 2021 February; 9(2):e001749. doi: 10.1136/jitc-2020-001749. PMID: 33589525; PMCID: PMC7887378.

In some embodiments, the antibody is the JTX-1811 as disclosed in Dépis, Fabien, et al. “Preclinical evaluation of JTX-1811, an anti-CCR8 antibody with enhanced ADCC activity, for preferential depletion of tumor-infiltrating regulatory T cells.” (2020): 4532-4532.

### CCR8 Depleting Antibodies

In some embodiments, the antibody suitable for depletion of CCR8 cancer cells mediates antibody-dependent cell-mediated cytotoxicity.

As used herein the term “antibody-dependent cell-mediated cytotoxicity” or “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein, the term “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

As used herein, the term “effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody suitable for depletion of cancer cells is a full-length antibody. In some embodiments, the full-length antibody is an IgG1 antibody. In some embodiments, the full-length antibody is an IgG3 antibody.

In some embodiments, the antibody suitable for depletion of cancer cells comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of cancer cells is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP1176195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian-like glycosylation pattern and capable of producing antibodies lacking fucose as glycosylation pattern (see for example EP1297172B1).

In some embodiments, the antibody suitable for depletion of cancer cells mediated complement dependant cytotoxicity.

As used herein, the term “complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody suitable for depletion of cancer cells mediates antibody-dependent phagocytosis.

As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

### CCR8 Multispecific Antibodies:

In some embodiments, the antibody suitable for depletion of CCR8 cancer cells is a multispecific antibody comprising a first antigen binding site directed against CCR8 and at least one second antigen binding site directed against an effector cell as above described. In said embodiments, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the multispecific antibody is thus a bispecific antibody.

In some embodiments, the bispecific antibody is a BiTE. As used herein, the term “Bispecific T-cell engager” or “BiTE” refers to a bispecific antibody that is a recombinant protein construct composed of two flexibly connected single-chain antibodies (scFv). One of said scFv antibodies binds specifically to a selected, target cell-expressed tumour antigen (i.e. CCR8), the second binds specifically to another molecule such as CD3, a subunit of the T-cell receptor complex on T cells. In some embodiments, the BiTE antibodies are capable of binding T cells transiently to target cells and, at the same time, activating the cytolytic activity of the T cells. The BiTE-mediated activation of the T cells requires neither specific T-cell receptors on the T cells, nor MHC I molecules, peptide antigens or co-stimulatory molecules on the target cell.

### CCR8 Antibody-Drug Conjugates:

In some embodiments, the antibody suitable for depletion of cancer cells is conjugated to a therapeutic moiety, i.e. a drug.

In some embodiments, the therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of cancer cells is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, momordica charantia inhibitor, curcin, crotin, sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of cancer cells is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (U.S. Pat. No. 5,663,149) and antifungal activity (Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of cancer cells is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al., Cancer Res 2010; 70(17): 6849-6858; Antonow D. et al., Cancer J 2008; 14(3): 154-169; Howard P. W. et al., Bioorg Med Chem Lett 2009; 19: 6463-6466 and Sagnou et al., Bioorg Med Chem Lett 2000; 10(18): 2083-2086.

In some embodiments, the antibody suitable for depletion of cancer cells is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody suitable for depletion of cancer cells is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of cancer cells is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

### CCR8 CAR-T Cells

In some embodiments, the agent is a CAR-T cell wherein the CAR comprises at least an extracellular antigen binding domain specific for CCR8.

In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the T cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28.

In some embodiments, the CAR comprises a chimeric fusion protein comprising an extracellular antigen binding domain specific for CCR8, a transmembrane domain and an intracellular signaling domain comprising a functional signaling domain derived from a stimulatory molecule. In some embodiments, the CAR comprises a chimeric fusion protein comprising an extracellular antigen binding domain specific for CCR8, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain specific for CCR8, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain specific for CCR8, a transmembrane domain and an intracellular signaling domain comprising at least two functional signaling domains derived from one or more costimulatory molecule(s) and a functional signaling domain derived from a stimulatory molecule.

In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

In particular aspects, CARs comprise fusions of single-chain variable fragments (scFv) derived from monoclonal antibodies that are specific for CCR8, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

In some embodiments, the chimeric antigen receptor of the present invention comprises at least one VH and/or VL sequence of an antibody that is specific for CCR8. In some embodiments, the portion of the CAR of the invention comprising an antibody or antibody fragment thereof that is specific for CCR8 may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment specific for CCR8. In a further aspect, the CAR comprises an antibody fragment that comprises a scFv that is specific for CCR8.

Methods for preparing CAR-T cells are well known in the art. In some embodiments, the cell (e.g., T cell) is transduced with a viral vector encoding a CAR. In some embodiments, the viral vector is a retroviral vector. In some embodiments, the viral vector is a lentiviral vector. In some embodiments, the cell may stably express the CAR. In some embodiments, the cell (e.g., T cell) is transfected with a nucleic acid, e.g., mRNA, cDNA, DNA, encoding a CAR. In some embodiments, the antigen binding domain of a CAR of the invention (e.g., a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In some embodiments, the chimeric antigen receptor of the present invention can be glycosylated, amidated, carboxylated, phosphorylated, esterified, N-acylated, cyclized via, e.g., a disulfide bridge, or converted into an acid addition salt and/or optionally dimerized or polymerized.

In some embodiments, the CAR activity can be controlled if desirable to optimize the safety and efficacy of a CAR therapy. There are many ways CAR activities can be regulated. For example, inducible apoptosis using, e.g., a caspase fused to a dimerization domain (see, e.g., Di et al., N Egnl. J. Med. 2011 Nov. 3; 365(18):1673-1683), can be used as a safety switch in the CAR therapy of the instant invention.

### Pharmaceutical Compositions:

Typically, the agent of the present invention is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

### Methods of Diagnosis

A further object of the present invention relates to a method of diagnosing a T-cell lymphoma in a patient comprising detecting the expression level of CCR8 in a sample obtained from the patient.

In some embodiments, the present invention relates to a method of diagnosing a T-cell lymphoma in a patient comprising detecting the expression level of CCR8 in a sample obtained from the patient, wherein an overexpression of CCR8 as compared to a predetermined reference value indicates that said patient suffers from said T-cell lymphoma.

In some embodiments, the method of the present invention is particularly suitable for diagnosing angioimmunoblastic T-cell lymphoma, hepatosplenic T-cell lymphoma, natural killer T-cell lymphoma or cutaneous T-cell lymphoma. In some embodiments, the method of the present invention is particularly suitable for diagnosing a cutaneous T-cell lymphoma. More particularly, the method of the present invention is particularly suitable for diagnosing a Sézary syndrome.

As used herein, the term “sample” refers to any biological sample obtained from the purpose of evaluation in vitro. In some embodiments, the sample is sample is a blood sample. In some embodiments, the sample is PBMC sample. In some embodiments, the sample is a sample of (i) purified blood leukocytes, (ii) peripheral blood mononuclear cells or PBMC, (iii) purified lymphocytes, (iv) purified T cells, (v) purified CD4+ T cells or (vi) purified CD3+ T cells. In some embodiments, the sample is a sample of purified CD3+CD4+CD26− and/or CD7− KIR3DL2+ lymphocytes. In some embodiments, the biological sample is a tissue sample. The term “tissue sample” includes sections of tissues such as biopsy or autopsy samples and frozen sections taken for histological purposes. Thus in some embodiments, the tissue sample may result from a biopsy performed in the subject's skin.

In some embodiments, the level of the marker is determined by immunohistochemistry (IHC). Immunohistochemistry typically includes the following steps i) fixing said tissue sample with formalin, ii) embedding said tissue sample in paraffin, iii) cutting said tissue sample into sections for staining, iv) incubating said sections with the binding partner specific for the marker, v) rinsing said sections, vi) incubating said section with a biotinylated secondary antibody and vii) revealing the antigen-antibody complex with avidin-biotin-peroxidase complex. Accordingly, the tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 125I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above descried), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In some embodiments, the level of the marker is determined by a flow-cytometric method. As used herein, the term “flow cytometric method” refers to a technique for counting cells of interest, by suspending them in a stream of fluid and passing them through an electronic detection apparatus. Flow cytometric methods allow simultaneous multiparametric analysis of the physical and/or chemical parameters of up to thousands of events per second, such as fluorescent parameters. Modern flow cytometric instruments usually have multiple lasers and fluorescence detectors. A common variation of flow cytometric techniques is to physically sort particles based on their properties, so as to purify or detect populations of interest, using “fluorescence-activated cell sorting”. As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest. Accordingly, FACS can be used with the methods described herein to isolate and detect the population of cells of the present invention. For example, fluorescence activated cell sorting (FACS) may be therefore used. involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. Flowjo, Laluza . . . ), data input devices, e.g., interface ports, a mouse, a keyboard, etc., fluid handling components, power sources, etc. More particularly, the sample is contacted with a panel of antibodies specific for the specific market of the population of cells of the interest. Such antibodies or antigen-binding fragments are available commercially from vendors such as R&D Systems, BD Biosciences, e-Biosciences, Biolegend, Proimmune and Miltenyi, or can be raised against these cell-surface markers by methods known to those skilled in the art. In some embodiments, an agent that specifically bind to a cell-surface marker, such as an antibody or antigen-binding fragment, is labelled with a tag to facilitate the isolation and detection of population of cells of the interest. As used herein, the terms “label” or “tag” refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods to isolate and detect the cancer cells. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650. The aforementioned assays may involve the binding of the antibodies to a solid support. The solid surface could be a microtitration plate coated with the antibodies. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, California).

In some embodiments, the method further comprises detecting the expression level of at least one further marker. Typically, the marker is selected from the group consisting of KIR3DL2, PLS3, Twist and NKp46.

In the present specification, the name of each of the various markers of interest refers to the internationally recognised name of the corresponding gene, as found in internationally recognised gene sequences and protein sequences databases, including in the database from the HUGO Gene Nomenclature Committee that is available notably at the following Internet address: http://www.gene.ucl.ac.uk/nomenclature/index.html. In the present specification, the name of each of the various markers of interest may also refer to the internationally recognised name of the corresponding gene, as found in the internationally recognised gene sequences and protein sequences database Genbank. Through these internationally recognised sequence databases, the nucleic acid and the amino acid sequences corresponding to each of the marker of interest described herein may be retrieved by the one skilled in the art.

Multiplex tissue analysis techniques are particularly useful for quantifying several markers in the tissue sample. Such techniques should permit at least five, or at least ten or more biomarkers to be measured from a single tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, MD).

In some embodiments, the L-IHC method can be performed on any of a variety of tissue samples, whether fresh or preserved. The samples included core needle biopsies that were routinely fixed in 10% normal buffered formalin and processed in the pathology department. Standard five μιη thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10μιη thick coated polymer backbone with 0.4μιη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, MD). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, MD). That technique is described in U.S. Pat. No. 7,473,532.

In some embodiments, the method of the present invention utilized the multiplex IHC technique developed by GE Global Research (Niskayuna, NY). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image—processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the subject, ii) TMA samples are then stained with anti-antibodies having specificity of the protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the level of the marker is determined at nucleic acid level. Typically, the level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

In some embodiments, the method of the present invention further comprises comparing the expression level of the marker with a predetermined reference value wherein detecting a difference between the expression level of the marker and the predetermined reference value indicates whether the subject has a T-cell lymphoma.

In some embodiments, the predetermined reference value is a relative to a number or value derived from population studies, including without limitation, subjects of the same or similar age range, subjects in the same or similar ethnic group, and subjects having the same severity of lesion. Such predetermined reference values can be derived from statistical analyses and/or risk prediction data of populations obtained from mathematical algorithms and computed indices. In some embodiments, retrospective measurement of the level of the marker in properly banked historical subject samples may be used in establishing these predetermined reference values. Accordingly, in some embodiments, the predetermined reference value is a threshold value or a cut-off value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the marker in a group of reference, one can use algorithmic analysis for the statistic treatment of the measured levels of the marker in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

Typically, as demonstrated in EXAMPLE, the expression level of the CCR8 is higher than the expression level determined in a sample from a healthy individual. In some embodiments, CCR8 expression level is determined with fluorescence intensity. In some embodiments, CCR8 expression level is determined with CCR8 mean fluorescence intensity. In some embodiments, the method comprises a further step consisting determining CCR8 mean fluorescence intensity and concluding that the patient suffers from a T-cell lymphoma when CCR8 mean fluorescence intensity is higher than 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. In some embodiments, the method comprises a further step consisting determining CCR8 mean fluorescence intensity and concluding that the patient suffers from a T-cell lymphoma when CCR8 mean fluorescence intensity is higher than 400. In some embodiments, CCR8 expression level is determined with CCR8 delta mean fluorescence intensity. In some embodiments, CCR8 delta mean fluorescence intensity is calculated as compared to an IgG2a control isotype expression level. In some embodiments, CCR8 delta mean fluorescence intensity is calculated as compared to an IgG2a control isotype mean fluorescence intensity. In some embodiments, the method comprises a further step consisting determining CCR8 delta mean fluorescence intensity and concluding that the patient suffers from a T-cell lymphoma when CCR8 delta mean fluorescence intensity is higher than 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. In some embodiments, the method comprises a further step consisting determining CCR8 delta mean fluorescence intensity and concluding that the patient suffers from a T-cell lymphoma when CCR8 delta mean fluorescence intensity is higher than 160.

Monitoring the influence of agents (e.g., drug compounds) on the level of expression CCR8 can be applied for monitoring the status of T-cell lymphoma in a patient with time. For example, the effectiveness of an agent to affect marker expression can be monitored during treatments of subjects receiving anti-T-cell lymphoma treatments.

Thus the present invention also provides a method for monitoring the effectiveness of treatment of a patient suffering from a T-cell lymphoma comprising the steps of:
(i) obtaining a pre-administration sample from a patient prior to administration of the agent;
(ii) detecting the level of expression of CCR8 in the pre-administration sample;
(iii) obtaining one or more post-administration samples from the patient;
(iv) detecting the level of expression of the same marker(s) in the post-administration samples;
(v) comparing the level of expression of CCR8 in the pre-administration sample with the level of expression of CCR8 in the post-administration sample or samples; and
(vi) altering the administration of the agent to the patient accordingly.

For example, a worse diagnosis that is determined by assessing the expression level of CCR8 during the course of treatment may indicate ineffective dosage and the desirability of increasing the dosage. Conversely, a better diagnosis that is determined by assessing the expression level of CCR8 may indicate efficacious treatment and no need to change dosage.

Accordingly, the present invention also relates to a method for adapting an therapy in a patient suffering from a T-cell lymphoma, wherein said method comprises the steps of:
a) performing, on at least one sample collected from said patient, the in vitro diagnosis method that is disclosed herein; and
b) adapting the therapy of said patient by administering to said patient.

The invention also relates to a kit for performing the diagnosis methods as described above. The kit comprises a plurality of reagents, in particular at least one agent that is capable of binding specifically to the CCR8 marker. Suitable reagents for binding with a marker protein include antibodies, antibody derivatives, antibody fragments, and the like. Suitable reagents for binding with a marker nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like. The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of the in vitro diagnosis method of the invention, and the like.

The invention will be further illustrated by the following figures and examples. However, these examples and figures should not be interpreted in any way as limiting the scope of the present invention.

### FIGURES

FIG. 1. CCR8 expression in fresh peripheral blood tumor cells from patients with Sézary syndrome

FIG. 2. Expression of CCR8 of T-cell lymphoma cell lines (SNK6, DERL-2 and HuT78 cell line)

FIG. 3. CCR8 is overexpressed at the cell surface of CTCL peripheral blood tumor cells and is involved in Sezary cell activation and proliferation. Flow cytometric analyses of CCR8 expression by peripheral blood Sezary cells, healthy controls T cells and T-cell lymphoma cell lines. (A) Gating strategy of peripheral blood Sezary cells (left panels) and mean fluorescence intensity of CCR8 expression in Sezary cells (versus control isotype) of 2 Sezary patients (right panels) (B) CCR8 delta mean fluorescence intensity (CCR8 mAb—control isotype) in fresh Sezary cells from Sezary patients compared to healthy controls T cells. (C) CCR8 stimulation by its CCL1 ligand induces Sezary cell proliferation. Fresh PBMC were incubated in CFSE and cultured over 96 h in IL-2 (100 IU/ml), CCL-1 (10 ng/ml) or IL-2/CCL1 and the percentage of CFSE^{lo} cells calculated among live KIR3DL2+ Sezary cells. (D) CCR8 expression in freshly isolated healthy controls peripheral blood mononuclear cells before and after 3 days of in vitro CD3/28 activation.

FIG. 4. (A) CCR8 expression on PDX cells from AITL. Splenocyte cells derived from patients with AITL were incubated with either control isotype or anti-human CD4 and anti-CCR8 antibodies (clone L263.G8) during 15 min at 4° C., then washed in PBS and analyzed on a LSRX20 flow cytometer. Histograms represent CCR8 expression on CD4+ cells. (B) CCR8 expression on HSTL cells. Splenocyte cells were stained with either control isotype or anti-CD3, CD5, TCRγ8 and anti-CCR8 antibodies. Histograms represent CCR8 expression on CD3+CD5−TCRγδ+ cells.

### EXAMPLE 1

### Methods

### CCR8 Expression in Fresh Peripheral Blood Tumor Cells From Patients With Sézary Syndrome

Study of CCR8 expression by flow cytometry on peripheral blood mononuclear cells of 4 patients with Sézary syndrome using anti-CD4, CD158k (=KIR3DL2, surface marker of Sézary cells), and CCR8 (CD198) antibodies (clone L263.G8) or control isotype after information and signature of informed consent.

### CCR8 Expression of T-Cell Lymphoma Cell Lines

Cells were incubated with control isotype or anti-CCR8 (CD198) antibody (clone L263.G8) during 15 min at 4° C., then washed in PBS and analyzed on a LSRX20 flow cytometer.

### Results

### CCR8 Expression in Fresh Peripheral Blood Tumor Cells From Patients With Sézary Syndrome

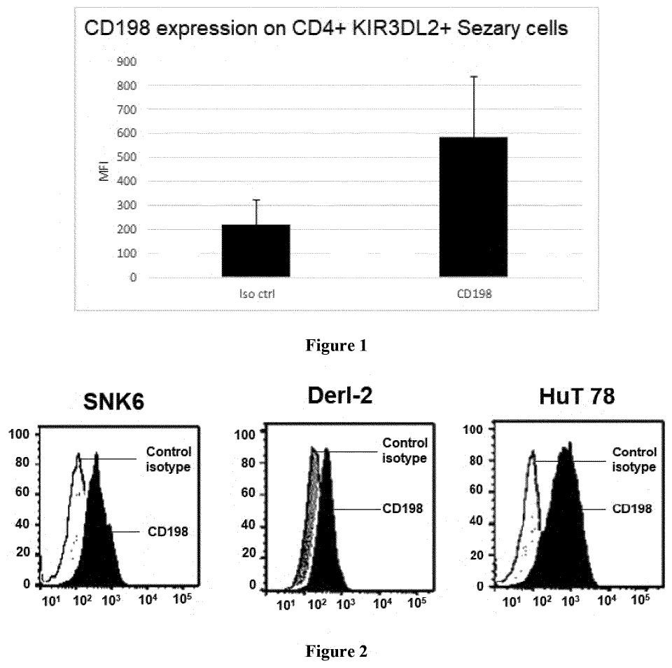

Overexpression of CCR8 (CD198) by circulating CD4+KIR3DL2+ tumor cells from patients with Sézary syndrome compared to reactive KIR3DL2−CD4 T cells (FIG. 1). Four different Sezary patient's cells were stained with anti-CD4, anti-KIR3DL2 and anti-CD198 antibodies. The CD198 expression was analyzed on the CD4+KIR3DL2+ tumor cell population.

### CCR8 Expression of T-Cell Lymphoma Cell Lines

SNK (EBV-positive NK/T cell lymphoma), DERL-2 (hepatosplenic gamma-delta T-cell lymphoma) and HuT78 (Sezary syndrome) cell lines were stained with anti-CCR8 antibody or control isotype and CCR8 expression was analyzed by flow cytometry (FIG. 2).

### EXAMPLE 2

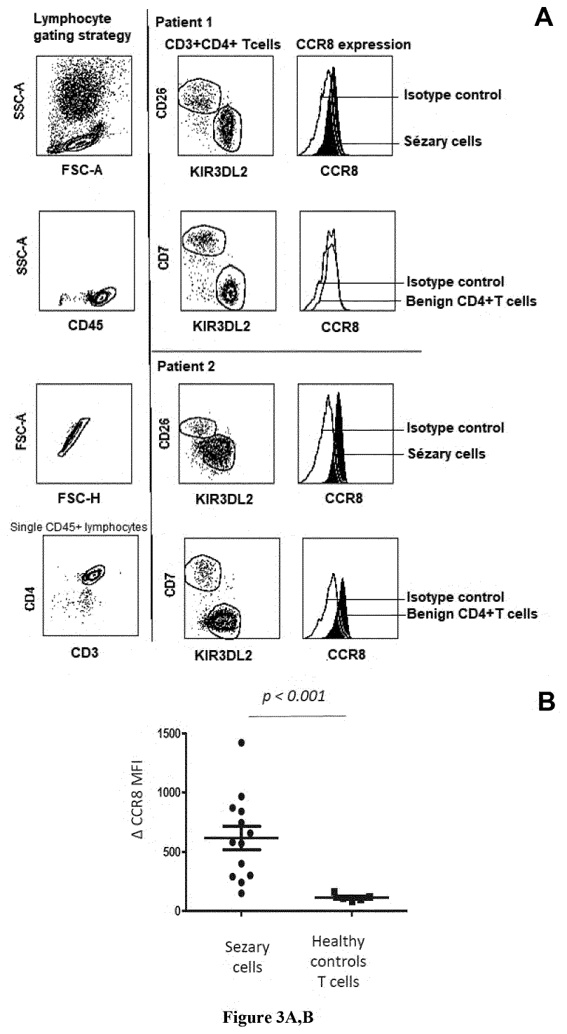

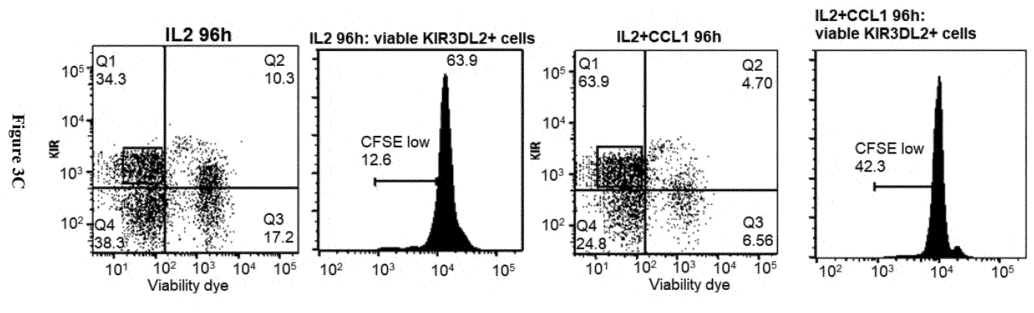

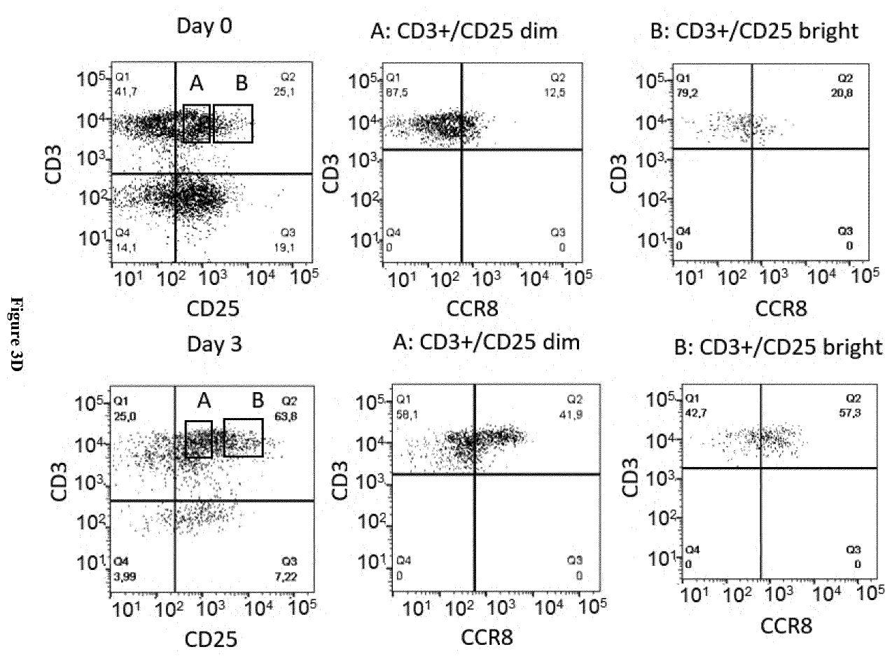

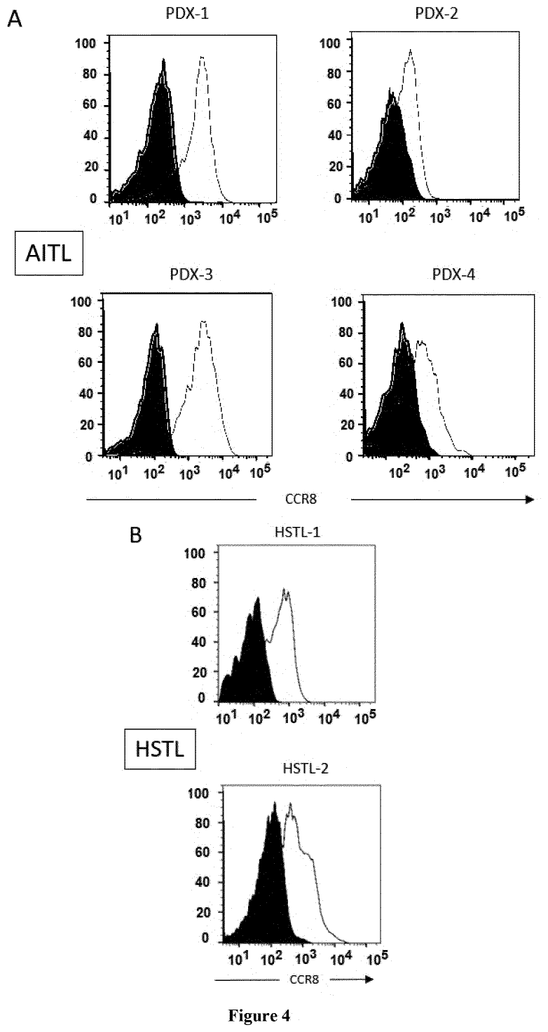

We performed flow cytometry analyses of peripheral blood leukocytes in 13 patients with SS and persistent blood involvement. Sézary cells were identified as CD3+CD4+CD26− and/or CD7−KIR3DL2+ lymphocytes as previously described (14,15) (FIG. 3A). CCR8 expression was measured using the L263G8 monoclonal antibody and control IgG2a isotype and compared to that of healthy donors' T cells. CCR8⁺ tumor T cells coexpressed CCR4 in all cases (data not shown). Peripheral blood CD4⁺CD25^{hi}CD127^{lo} Tregs from Sézary patients did not express high levels of CCR8 (data not shown). CCR8 delta median mean fluorescence intensity (CCR8 mAb—control isotype) was 580 (range, 150-1420) in Sezary cells versus 110 (range, 80-160) in healthy controls (p<0.001, FIG. 3B). Interestingly, not only CTCL HuT78 (SS) cell line but also the NK/T cell lymphoma SNK6, hepatosplenic gamma-delta T cell lymphoma DERL-2 cell lines and AITL cell lines expressed CCR8 (FIG. 2 and FIG. 4), suggesting CCR8 as a potential therapeutic target in different T-cell lymphoma subtypes. CCR8 engagement by its ligand CCL18 and CCL1 induced a significant Erk1/2 phosphorylation at 30 minutes and was independent on IL-2 in tumor cells of Sézary patients (data not shown). Moreover, it seems in some patients that CCL1 together with IL-2 induced a higher Sezary cell proliferation as compared to IL-2 alone (42% versus 12% CFSE^{lo} Sezary cells) (FIG. 3C). The CCR8 expression was also analyzed after in vitro activation of healthy controls' freshly isolated peripheral blood lymphocytes before (day 0) or after CD3/28 activation (day 3). CCR8 expression by T cells was significantly increased after 3 days of in vitro activation and was higher in CD25^{bright} activated T cells compared to CD25^{int} T cells (FIG. 3D)

### Conclusion

In conclusion, this study confirms the overexpression of the homing marker CCR8 by peripheral blood Sézary cells compared to healthy controls T cells. Since this molecule is also expressed at the cell-surface on other T-cell lymphoma cell lines, our results suggest that CCR8 might be a therapeutic target in distinct aggressive T-cell lymphoma subtypes.

Our study is the first to analyze CCR8 as a potential therapeutic target in CTCL. Immunomodulatory treatments (16) such as PD-1 inhibition (7), or allogeneic stem cell transplantation (17) have shown able to produce long-term responses in CTCL, suggesting that the activation of antitumor immune responses might provide long-lasting disease control. In mogamulizumab-treated patients, depletion of CCR4-expressing peripheral activated Tregs was associated with immune side effects but these immune reactions were associated with disease response and long-term disease control (5,6,18). CCR8 has recently been proposed as optimal tumor Treg target (19). Unlike CCR4, CCR8 was selectively expressed on human tumor Tregs and minimally expressed on proinflammatory effector T cells. Preclinical mouse tumor models showed that depletion of CCR8+ Tregs through an FcyR-engaging anti-CCR8 antibody enabled dose-dependent, effective, and long-lasting antitumor immunity that synergized with PD-1 blockade (19). Fc-optimized, nonfucosylated anti-human CCR8 antibodies specifically depleted Tregs and not effector T cells in ex vivo tumor cultures from primary human specimens (19).

### REFERENCES

Throughout this application, various references describe the state of the art to which this invention pertains. The disclosures of these references are hereby incorporated by reference into the present disclosure.
1. Bagot M, Moretta A, Sivori S, Biassoni R, Cantoni C, Bottino C, et al. CD4(+) cutaneous T-cell lymphoma cells express the p140-killer cell immunoglobulin-like receptor. Blood. 2001; 97(5): 1388-91.
2. Battistella M, Leboeuf C, Ram-Wolff C, Hurabielle C, Bonnafous C, Sicard H, et al. KIR3DL2 expression in cutaneous T-cell lymphomas: expanding the spectrum for KIR3DL2 targeting. Blood. 2017; 130(26):2900-2.
3. Kim YH, Bagot M, Pinter-Brown L, Rook AH, Porcu P, Horwitz SM, et al. Mogamulizumab versus vorinostat in previously treated cutaneous T-cell lymphoma (MAVORIC): an international, open-label, randomised, controlled phase 3 trial. Lancet Oncol. 2018; 19(9):1192-204.
4. Sugiyama D, Nishikawa H, Maeda Y, Nishioka M, Tanemura A, Katayama I, et al. Anti-CCR4 mAb selectively depletes effector-type FoxP3+CD4+ regulatory T cells, evoking antitumor immune responses in humans. Proc Natl Acad Sci USA. 2013; 110(44):17945-50.
5. Bonnet P, Battistella M, Roelens M, Ram-Wolff C, Herms F, Frumholtz L, et al. Association of autoimmunity and long-term complete remission in patients with Sézary syndrome treated with mogamulizumab. Br J Dermatol. 2019; 180(2):419-20.
6. Algarni AS, Ram-Wolff C, Bagot M, De Masson A. Mogamulizumab-induced vitiligo in patients with Sézary syndrome: three cases. Eur J Dermatol. 2021; 31(2):213-6.
7. Khodadoust MS, Rook AH, Porcu P, Foss F, Moskowitz AJ, Shustov A, et al. Pembrolizumab in Relapsed and Refractory Mycosis Fungoides and Sézary Syndrome: A Multicenter Phase II Study. J Clin Oncol. 2020; 38(1):20-8.
8. Bensussan A, Janela B, Thonnart N, Bagot M, Musette P, Ginhoux F, et al. Identification of CD39 as a Marker for the Circulating Malignant T-Cell Clone of Sézary Syndrome Patients. J Invest Dermatol. 2019; 139(3):725-8.
9. Jariwala N, Benoit B, Kossenkov AV, Oetjen LK, Whelan TM, Cornejo CM, et al. TIGIT and Helios Are Highly Expressed on CD4+ T Cells in Sézary Syndrome Patients. J Invest Dermatol. 2017; 137(1):257-60.
10. McCully ML, Ladell K, Hakobyan S, Mansel RE, Price DA, Moser B. Epidermis instructs skin homing receptor expression in human T cells. Blood. 2012; 120(23):4591-8.
11. McCully ML, Ladell K, Andrews R, Jones RE, Miners KL, Roger L, et al. CCR8 Expression Defines Tissue-Resident Memory T Cells in Human Skin. J Immunol Baltim. 2018; 200(5): 1639-50.
12. Clark RA, Watanabe R, Teague JE, Schlapbach C, Tawa MC, Adams N, et al. Skin effector memory T cells do not recirculate and provide immune protection in alemtuzumab-treated CTCL patients. Sci Transl Med. 2012; 4(117): 117ra7.
13. Van Damme H, Dombrecht B, Kiss M, Roose H, Allen E, Van Overmeire E, et al. Therapeutic depletion of CCR8+ tumor-infiltrating regulatory T cells elicits antitumor immunity and synergizes with anti-PD-1 therapy. J Immunother Cancer. 2021; 9(2).
14. Moins-Teisserenc H, Daubord M, Clave E, Douay C, Félix J, Marie-Cardine A, et al. CD158k is a reliable marker for diagnosis of Sézary syndrome and reveals an unprecedented heterogeneity of circulating malignant cells. J Invest Dermatol. 2015; 135(1): 247-57.
15. Roelens M, de Masson A, Ram-Wolff C, Maki G, Cayuela J-M, Marie-Cardine A, et al. Revisiting the initial diagnosis and blood staging of mycosis fungoides and Sézary syndrome with the KIR3DL2 marker. Br J Dermatol. 2020; 182(6): 1415-22.
16. Roccuzzo G, Giordano S, Fava P, Pileri A, Guglielmo A, Tonella L, et al. Immune Check Point Inhibitors in Primary Cutaneous T-Cell Lymphomas: Biologic Rationale, Clinical Results and Future Perspectives. Front Oncol. 2021; 11:733770.
17. de Masson A, Beylot-Barry M, Bouaziz J-D, Peffault de Latour R, Aubin F, Garciaz S, et al. Allogeneic stem cell transplantation for advanced cutaneous T-cell lymphomas: a study from the French Society of Bone Marrow Transplantation and French Study Group on Cutaneous Lymphomas. Haematologica. 2014; 99(3):527-34.
18. Trum NA, Zain J, Martinez XU, Parekh V, Afkhami M, Abdulla F, et al. Mogamulizumab Efficacy is Underscored by its Associated Rash that Mimics Cutaneous T-cell Lymphoma: A Retrospective Single-Centre Case Series. Br J Dermatol. 2021 (Epub ahead of print)
19. Campbell JR, McDonald BR, Mesko PB, Siemers NO, Singh PB, Selby M, et al. Fc-Optimized Anti-CCR8 Antibody Depletes Regulatory T Cells in Human Tumor Models. Cancer Res. 2021; 81(11): 2983-94.

1. A method of treating a T-cell lymphoma in a patient in need thereof comprising administering to the patient a therapeutically effective amount of an agent capable of inducing cell death of CCR8 expressing cancer cells.

2. The method of claim 1 wherein the T-cell lymphoma is angioimmunoblastic T-cell lymphoma, hepatosplenic T-cell lymphoma, natural killer T-cell lymphoma or cutaneous T-cell lymphoma.

3. The method of claim 1 wherein the T-cell lymphoma is cutaneous T-cell lymphoma.

4. The method of claim 3 wherein the T-cell lymphoma is Sézary syndrome.

5. The method of claim 1 wherein the agent is a CCR8 inhibitor.

6. The method of claim 1 wherein the agent is an antibody having binding affinity for CCR8.

7. The method of claim 6 wherein the agent is an antibody directed against at least one extracellular domain of CCR8 and leads to the depletion of CCR8 expression cancer cells.

8. The method of claim 7 wherein the antibody suitable for depletion of CCR8 cancer cells mediates antibody-dependent cell-mediated cytotoxicity.

9. The method of claim 7 wherein the antibody is a multispecific antibody comprising a first antigen binding site directed against CCR8 and at least one second antigen binding site directed against an effector cell.

10. The method of claim 7 wherein the antibody is conjugated to a cytotoxic moiety.

11. The method of claim 1 wherein the agent is a CAR-T cell wherein the CAR comprises at least an extracellular antigen binding domain specific for CCR8.

12. A method of diagnosing a T-cell lymphoma in a patient comprising detecting the expression level of CCR8 in a sample obtained from the patient.

13. The method of claim 12 for diagnosing angioimmunoblastic T-cell lymphoma, hepatosplenic T-cell lymphoma, natural killer T-cell lymphoma or cutaneous T-cell lymphoma.

14. The method of claim 12 for diagnosing a cutaneous T-cell lymphoma.

15. The method of claim 14 for diagnosing a Sézary syndrome.

16. The method of claim 14 that further comprises detecting the expression level of at least one further marker selected from the group consisting of KIR3DL2, PLS3, Twist and NKp46.


SEQUENCE LISTING (1 sequence)

SEQ ID NO: 1 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL